(19) United States
(12) Patent Application Publication
(10) Pub. No.: US 2009/0028807 A1
(43) Pub. Date: Jan. 29, 2009

(54) COMPOSITION COMPRISING A POLYCONDENSATE, METHOD OF TREATMENT, POLYCONDENSATE, AND METHOD OF PREPARATION

(75) Inventors: Pascal GIUSTINIANI, La Garenne Colombes (FR); Gerard Malle, Villiers S/Morin (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/142,413

(22) Filed: Jun. 19, 2008

Related U.S. Application Data
(60) Provisional application No. 60/990,302, filed on Nov. 27, 2007.
(60) Provisional application No. 60/948,495, filed on Jul. 9, 2007.

(30) Foreign Application Priority Data
Jun. 21, 2007 (FR) 07 55919
Oct. 4, 2007 (FR) 07 58059

(51) Int. Cl. A61K 8/36; A61K 47/12; A61Q 5/00; A61Q 19/04

(52) U.S. Cl. 424/60; 514/784; 424/70.1

(57) ABSTRACT

The present application relates to a cosmetic or pharmaceutical, in particular lipstick, composition comprising a polycondensate The application also relates to a method of cosmetic treatment using the composition, the polycondensate thus defined and a method of preparing the polycondensate.

### REFERENCE TO PRIOR APPLICATIONS

This application claims priority to U.S. provisional applications 60/948,495 filed Jul. 9, 2007, and 60/990,302, filed Nov. 27, 2007; and to French patent applications 0755919 filed Jun. 21, 2007, and 0758059 filed Oct. 4, 2007, both incorporated herein by reference.

### FIELD OF THE INVENTION

The present invention relates to novel polymers of the polycondensate family of the modified alkyd type, and to their use in, e.g., cosmetic compositions, in particular in lipsticks, to their use in pharmaceutical compostions, to cosmetic and pharmaceutical compositions containing them, and to methods for preparing the polycondensates.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Numerous cosmetic compositions exist for which gloss properties of the film deposited, after application to keratin materials (skin, lips, superficial body growths), are desired. There may be mentioned, for example, lipsticks, nail varnishes or certain hair products.

In order to obtain such a result, it is possible to combine specific raw materials, in particular lanolins, with so-called glossy oils, such as polybutenes which have nevertheless a high viscosity; or esters of a fatty acid or a fatty alcohol in which the number of carbons is high; or alternatively certain vegetable oils; or else esters resulting from the partial or complete esterification of a hydroxylated aliphatic compound with an aromatic acid, as described in patent application EP 1097699.

It is also known to combine lanolins with polyesters obtained by sequential reaction of castor oil with isostearic acid and then with succinic acid, as described in U.S. Pat. No. 6,342,527.

To improve the gloss of the deposited film, and its staying power, it has also been proposed to use esters resulting from the condensation of a polyol with a “new” type carboxylic acid, in particular in FR 2838049.

There may also be mentioned EP 1457201, which describes a composition combining a polyester of triglycerides of hydroxylated carboxylic acids and an oil of low molecular mass chosen from polybutylenes, hydrogenated polyisobutylenes, hydrogenated or non-hydrogenated polydecenes, copolymers of vinylpyrrolidones, esters of linear fatty acids, hydroxylated esters, esters of branched C24-C28 fatty alcohols or fatty acids, silicone oils and/or oils of plant origin.

In patent application EP 0792637, a composition combining an aromatic ester or a polymer of the polybutene or polyisobutene type is described.

In patent application EP 1155687, a process is described which consists in incorporating, into an oily phase constituted of a cosmetically acceptable oil, an organopolysiloxane having at least 2 groups capable of establishing hydrogen bonds.

However, these compositions and combinations, even though they significantly improve the gloss, are still judged inadequate for the purpose of a long staying power of this gloss, over time.

### DETAILED DESCRIPTION OF THE PREFERRED EMBODIMENTS

One aim of the present invention is to provide novel polymers which confer a significant gloss on a deposit, in particular a film-forming deposit, while maintaining good stability of this gloss over time; it may find a particularly advantageous application in the field of, e.g., pharmaceuticals and cosmetics such as lipsticks. Moreover, polymers are also sought which can additionally advantageously confer on the composition excellent staying power over time on keratin materials, in particular on the lips.

For this purpose, the inventors have now provided novel alkyd-type polycondensates having the desired properties.

Alkyd resins constitute a particular class of polyesters in being the product of reaction of polyols and polycarboxylic acids, generally modified by unsaturated fatty acids, such as oleic acid, or by unsaturated oils, for example soybean or castor oil, which make it possible to modulate their film-forming properties, in particular their rate of drying, their hardness and their resistance.

Thus, there has been proposed in the document U.S. Pat. No. 2,915,488 modified alkyd resins in which part of the fatty acids derived from soybean oil has been replaced by benzoic acid. These novel resins have improved properties in terms of resistance to alkalis and to detergents; the films containing them dry more rapidly and are harder. However, no application, in particular no cosmetic or topical application, has been envisaged for these resins.

Moreover, the fatty acids present in soybean oil are predominantly constituted of two unsaturated fatty acids: about 55% of linoleic acid (C18:2) and 28% of oleic acid (C18:1), according to “Surface Coatings Science and Technology”, 2^{nd} edition, John Wiley & Sons, pages 104 and 105. Now, it is known that certain unsaturated fatty acids can undergo, over time, auto-oxidation which may be responsible for the phenomena of rancidity, which can result in problems for preservation of compositions comprising these raw materials. Furthermore, the alkyd resins described in U.S. Pat. No. 2,915,488, which comprise a high proportion of linoleic and oleic fatty acids, are not optimum, in particular in terms of stability, for use in cosmetics.

The crosslinking of alkyd resins by oxidation in air with formation of hydroperoxides plays a key role in the rate of drying of the film and, consequently, in its final properties of hardness and resistance to external attacks. In the usual fields of application of these alkyd resins, namely paints, the rate of crosslinking, and therefore of drying, is generally accelerated by the addition of particular metal salts, called “dryers”, such as cobalt naphthenates and octanoates which accelerate the decomposition of hydroperoxides; this is in particular described in Principles of Polymerization, 4^{th} edition, John Wiley & Sons, pages 737-738 and also in Surface Coatings Science and Technology, 2^{nd} edition, John Wiley & Sons, Tables 2.3 and 2.4 pages 526-530.

However, the use of such metal salts is obviously not desirable in cosmetics or pharmaceuticals for obvious reasons of toxicity.

Moreover, most alkyd resins do not have suitable solubility in the oily media normally used in cosmetics, such as vegetable oils, alkanes, fatty esters, fatty alcohols, silicone oils, and in particular comprising isododecane, Parleam, isononyl isononanoate, octyldodecanol, phenyltrimethicone, C12-C15 alkyl benzoate and/or D5 (decamethylcyclopentasiloxane).

After major research studies, the inventors have discovered surprisingly and unexpectedly that certain polycondensates with a higher content of particular carboxylic acids, including non-aromatic monocarboxylic acids, lead to improved performance in terms of gloss, maintenance of the gloss, and additionally long staying power of the film obtained, while being capable of being carried in customary cosmetic media, in particular the customary cosmetic oily media.

One subject of the present invention is therefore a cosmetic or pharmaceutical composition comprising, optionally but preferably in a cosmetically or pharmaceutically acceptable medium, at least one polycondensate capable of being obtained, or obtained, by the reaction:
of 10 to 30% by weight, relative to the total weight of the polycondensate, of at least one polyol comprising 3 to 6 hydroxyl groups;
of 22 to 80% by weight, relative to the total weight of the polycondensate, of at least one linear, branched and/or cyclic, saturated or unsaturated, non-aromatic monocarboxylic acid comprising 10 to 32 carbon atoms, and having a melting point greater than or equal to 25° C.;
of 0.1 to 35% by weight, relative to the total weight of the polycondensate, of at least one linear, branched and/or cyclic, saturated or unsaturated, non-aromatic monocarboxylic acid comprising 6 to 32 carbon atoms and having a melting point strictly less than 25° C.;
of 0.1 to 10% by weight, relative to the total weight of the polycondensate, of at least one aromatic monocarboxylic acid comprising 7 to 11 carbon atoms, optionally additionally substituted with 1 to 3 linear, branched and/or cyclic, saturated or unsaturated alkyl radicals which comprise 1 to 32 carbon atoms;
of 1 to 40% by weight, relative to the total weight of the polycondensate, of at least one linear, branched and/or cyclic, saturated or unsaturated, or even aromatic, polycarboxylic acid comprising at least 2 carboxyl groups COOH, which may comprise heteroatoms; and/or a cyclic anhydride of such a polycarboxylic acid; and/or a lactone comprising at least one COOH group.

Another subject of the invention is a polycondensate which may be obtained, or is obtained, by the reaction:
of 10 to 30% by weight, relative to the total weight of the polycondensate, of at least one polyol comprising 3 to 6 hydroxyl groups;
of 22 to 80% by weight, relative to the total weight of the polycondensate, of at least one linear, branched and/or cyclic, saturated or unsaturated, non-aromatic monocarboxylic acid comprising 10 to 32 carbon atoms, and having a melting point greater than or equal to 25° C.;
of 0.1 to 35% by weight, relative to the total weight of the polycondensate, of at least one linear, branched and/or cyclic, saturated or unsaturated, non-aromatic monocarboxylic acid comprising 6 to 32 carbon atoms and having a melting point strictly less than 25° C.;
of 0.1 to 10% by weight, relative to the total weight of the polycondensate, of at least one aromatic monocarboxylic acid comprising 7 to 11 carbon atoms, optionally additionally substituted with 1 to 3 linear, branched and/or cyclic, saturated or unsaturated alkyl radicals which comprise 1 to 32 carbon atoms;
of 1 to 40% by weight, relative to the total weight of the polycondensate, of at least one linear, branched and/or cyclic, saturated or unsaturated, or even aromatic, polycarboxylic acid comprising at least 2 carboxyl groups COOH, in particular 2 to 4 COOH groups, and which may comprise one or more heteroatoms; and/or a cyclic anhydride of such a polycarboxylic acid; and/or a lactone comprising at least one COOH group.

Finally, the polycondensate therefore comprises these monomers, in the proportions indicated; most preferably the polycondensate is constituted (exclusively or solely comprises) of these monomers.

In particular, the cosmetic and pharmaceutical compositions exhibit good applicability and good covering power; good adhesion to the support, whether on a tablet, the nail, the hair, the eyelashes, the skin or the lips; adequate flexibility and resistance of the film, so as to avoid cracks, for example in the case of lipsticks or varnish; and also an excellent level of long-lasting gloss. The comfort and slip properties are also very satisfactory. These polycondensates can be easily carried in cosmetic solvent or oily media, in particular oils, fatty alcohols and/or fatty esters, which facilitates their use in the cosmetic field, in particular in lipsticks or foundations.

The polycondensates according to the invention may be easily prepared, in a single synthesis step, and without producing waste, at low cost.

Moreover, it is possible to easily modify the structure and/or the properties of the polycondensates according to the invention by varying the chemical nature of the various constituents and/or their proportions.

The polycondensates according to the invention are advantageously branched; it is possible to think that this makes it possible to generate a network by entanglement of the polymer chains, and to therefore obtain the desired properties, in particular in terms of improved staying power, improved gloss, and in terms of solubility. It has indeed been observed that linear polycondensates do not make it possible to obtain a notable improvement in the staying power of the composition, and that polycondensates of the dendrimer type, whose chains are regular, do not have optimum solubility.

The polycondensates according to the invention are polycondensates of the alkyd type, and are therefore capable of being obtained, or obtained, by esterification/polycondensation, according to methods known to a person skilled in the art, of the constituents described below.

One of the constituents useful for the preparation of the polycondensates according to the invention is a compound comprising 3 to 6 hydroxyl (polyol) groups, in particular 3 to 4 hydroxyl groups. It is quite obviously possible to use a mixture of such polyols.

The polyol may in particular be a saturated or unsaturated, linear, branched and/or cyclic carbon-containing, in particular hydrocarbon-containing, compound comprising 3 to 18 carbon atoms, in particular 3 to 12, even 4 to 10 carbon atoms, and 3 to 6 hydroxyl (OH) groups, and capable of additionally comprising one or more oxygen atoms intercalated in the chain (ether functional group).

The polyol is preferably a linear or branched, saturated hydrocarbon compound comprising 3 to 18 carbon atoms, in particular 3 to 12, even 4 to 10 carbon atoms, and 3 to 6 hydroxyl (OH) groups.

It may be chosen from, alone or as a mixture:
triols, such as 1,2,4-butanetriol, 1,2,6-hexanetriol, trimethylolethane, trimethylolpropane, glycerol;
tetraols, such as pentaerythritol (tetramethylolmethane), erythritol, diglycerol or ditrimethylolpropane;
pentols such as xylitol;
hexyls such as sorbitol and mannitol; or alternatively dipentaerythritol or triglycerol.

Preferably, the polyol is chosen from glycerol, diglycerol, pentaerythritol, sorbitol and mixtures thereof; and better still is pentaerythritol.

The polyol, or the polyol mixture, preferably represents 10 to 30% by weight, in particular 12 to 25% by weight, and even better 14 to 22% by weight, of the total weight of the final polycondensate.

Another constituent useful for the preparation of the polycondensates according to the invention is a linear, branched and/or cyclic, saturated or unsaturated, non-aromatic monocarboxylic acid comprising 10 to 32 carbon atoms, in particular 12 to 28 carbon atoms and better still 12 to 24 carbon atoms; and having a melting point greater than or equal to 25° C., in particular greater than or equal to 28° C., or even 30° C. It is quite obviously possible to use a mixture of such non-aromatic monocarboxylic acids.

It has been observed that when such an acid is used, in the quantities indicated, it is possible, on the one hand, to obtain good gloss and staying power for the gloss and, on the other hand, to reduce the quantity of waxes customarily present in the composition envisaged.

The expression non-aromatic monocarboxylic acid is understood to mean a compound of formula RCOOH, in which R is a linear, branched and/or cyclic, saturated or unsaturated hydrocarbon radical comprising 9 to 31 carbon atoms, in particular 11 to 27 carbon atoms, and better still 11 to 23 carbon atoms.

Preferably, the R radical is saturated. Better still, the R radical is linear or branched, and is preferably C11-C21.

Among the non-aromatic monocarboxylic acids having a melting point greater than or equal to 25° C., which are liable to be used, there may be mentioned, alone or as a mixture:
among the saturated monocarboxylic acids: decanoic (capric) acid, lauric acid, tridecanoic acid, myristic acid, palmitic acid, stearic acid, arachidonic acid, behenic acid, cerotic acid (hexacosanoic) acid;
among the unsaturated but non-aromatic monocarboxylic acids: petroselinic acid, vaccenic acid, elaidic acid, gondoic acid, gadoleic acid, erucic acid, nervonic acid.

Preferably, it is possible to use lauric acid, palmitic acid, stearic acid, behenic acid and mixtures thereof, and better still stearic acid or behenic acid, alone.

The non-aromatic monocarboxylic acid having a melting point greater than or equal to 25° C., or the mixture of the acids, preferably represents 22 to 80% by weight, in particular 25 to 75% by weight, or even 27 to 70% by weight, and even better 28 to 65% by weight, of the total weight of the final polycondensate.

Another constituent useful for the preparation of the polycondensates according to the invention is a linear, branched and/or cyclic, saturated or unsaturated, non-aromatic monocarboxylic acid comprising 6 to 32 carbon atoms, in particular 8 to 28 carbon atoms and better still 10 to 20, or even 12 to 18, carbon atoms; and having a melting point strictly less than 25° C., in particular less than 20° C., or even 15° C. It is quite obviously possible to use a mixture of such non-aromatic monocarboxylic acids.

The expression non-aromatic monocarboxylic acid is understood to mean a compound of formula RCOOH, in which R is a linear, branched and/or cyclic, saturated or unsaturated hydrocarbon radical comprising 5 to 31 carbon atoms, in particular 7 to 27 carbon atoms, and better still 9 to 19 carbon atoms, or even 11 to 17 carbon atoms.

Preferably, the R radical is saturated. Better still,

the R radical is linear or branched, and is preferably C5-C31.

Among the non-aromatic monocarboxylic acids having a melting point of less than 25° C., liable to be used, there may be mentioned, alone or as a mixture:
among the saturated monocarboxylic acids: caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, isononanoic acid, isostearic acid;
among the unsaturated but non-aromatic monocarboxylic acids: caproleic acid, obtusilic acid, undecylenic acid, dodecylenic acid, linderic acid, myristoleic acid, physeteric acid, tsuzuic acid, palmitoleic acid, oleic acid, linoleic acid, linolenic acid, arachidonic acid.

Preferably, it is possible to use isooctanoic acid, isononanoic acid, isostearic acid, and mixtures thereof, and better still isostearic acid alone.

The non-aromatic monocarboxylic acid having a melting point of less than 25° C., or the mixture of the acids, preferably represents 0.1 to 35% by weight, in particular 0.5 to 32% by weight, or even 1 to 30% by weight, and even better 2 to 28% by weight, of the total weight of the final polycondensate.

Preferably, the total quantity of non-aromatic monocarboxylic acids, namely those having a melting point greater than 25° C.+those having a melting point of less than 25° C., is advantageously between 30 and 80% by weight, in particular between 40 and 70% by weight, or even 45 and 65% by weight, and better still between 50 and 60% by weight, of the total weight of the final polycondensate.

Another constituent useful for the preparation of the polycondensates according to the invention is an aromatic monocarboxylic acid comprising 7 to 11 carbon atoms, optionally additionally substituted with 1 to 3 linear, branched and/or cyclic, saturated or unsaturated alkyl radicals which comprise 1 to 32 carbon atoms, in particular 2 to 12, or even 3 to 8 carbon atoms.

It is of course possible to use a mixture of such aromatic monocarboxylic acids.

The expression aromatic monocarboxylic acid is understood to mean a compound of the formula R′COOH, in which R′ is an aromatic hydrocarbon radical comprising 6 to 10 carbon atoms, and in particular the benzoic and naphthoic radicals. The R′ radical may be additionally substituted with 1 to 3 linear, branched and/or cyclic, saturated or unsaturated alkyl radicals comprising 1 to 32 carbon atoms, in particular 2 to 12, or even 3 to 8 carbon atoms; and in particular chosen from methyl, ethyl, propyl, isopropyl, butyl, isobutyl, tert-butyl, pentyl, isopentyl, neopentyl, cyclopentyl, hexyl, cyclohexyl, heptyl, isoheptyl, octyl or isooctyl.

Among the aromatic monocarboxylic acids liable to be used, there may be mentioned, alone or as a mixture, benzoic acid, o-toluic acid, m-toluic acid, p-toluic acid, 1-naphthoic acid, 2-naphthoic acid, 4-tert-butylbenzoic acid, 1-methyl-2-naphthoic acid, 2-isopropyl-1-naphthoic acid.

Preferably, it is possible to use benzoic acid, 4-tert-butylbenzoic acid, o-toluic acid, m-toluic acid, 1-naphthoic acid, alone or as mixtures; and better still benzoic acid alone.

The aromatic monocarboxylic acid, or the mixture of the acids, preferably represents 0.1 to 10% by weight, in particular 0.5% to 9.95% by weight, better still from 1 to 9.5% by weight, or even 1.5 to 8% by weight, of the total weight of the final polycondensate.

Another constituent useful for the preparation of the polycondensates according to the invention is a polycarboxylic acid, and/or a cyclic anhydride of such a polycarboxylic acid, and/or a lactone carrying at least one COOH group; and mixtures thereof.

The polycarboxylic acid may in particular be chosen from saturated or unsaturated, or even aromatic, linear, branched and/or cyclic polycarboxylic acids comprising 2 to 50, in particular 2 to 40, carbon atoms, in particular 3 to 36, or even 3 to 18, and better still 4 to 12 carbon atoms, or even 5 to 10 carbon atoms; the acid comprising at least two carboxyl groups COOH, preferably 2 to 4 COOH groups; and capable of comprising 1 to 10, preferably 1 to 6, heteroatoms, which are identical or different, chosen from O, N and S; and/or capable of comprising at least one perfluorinated radical chosen from —CF₂— (divalent) or —CF₃.

Preferably, the polycarboxylic acid is linear, saturated and aliphatic, and comprises 2 to 36 carbon atoms, in particular 3 to 18 carbon atoms, or even 4 to 12 carbon atoms; or else is aromatic and comprises 8 to 12 carbon atoms. It preferably comprises 2 to 4 COOH groups.

The cyclic anhydride of such a polycarboxylic acid may in particular correspond to one of the following formulae:

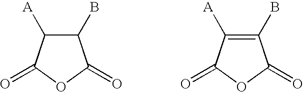

in which the A and B groups are, independently of each other:
a hydrogen atom,
a linear, branched and/or cyclic, saturated or unsaturated, aliphatic or aromatic carbon-containing radical comprising 1 to 16 carbon atoms, in particular 2 to 10 carbon atoms, or even 4 to 8 carbon atoms, in particular methyl or ethyl;
or else A and B taken together form a saturated or unsaturated, or even aromatic, ring comprising in total 5 to 14, in particular 5 to 10, or even 6 to 7 carbon atoms.

Preferably, A and B represent a hydrogen atom or form together an aromatic ring comprising in total 6 to 10 carbon atoms.

Among the polycarboxylic acids or their anhydrides, which are liable to be used, there may be mentioned, alone or as a mixture:
dicarboxylic acids such as decanedioic acid, dodecanedioic acid, cyclopropanedicarboxylic acid, cyclohexanedicarboxylic acid, cyclobutanedicarboxylic acid, 1,4-naphthalenedicarboxylic acid, 2,3-naphthalenedicarboxylic acid, 2,6-naphthalenedicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid, dimers of fatty acids (in particular as C36) such as the products marketed under the names Pripol 1006, 1009, 1013 and 1017, by Uniqema;
tricarboxylic acids such as cyclohexanetricarboxylic acid, trimellitic acid, 1,2,3-benzenetricarboxylic acid, 1,3,5-benzenetricarboxylic acid;
tetracarboxylic acids such as butanetetracarboxylic acid and pyrromellitic acid,
the cyclic anhydrides of these acids and in particular phthalic anhydride, trimellitic anhydride, maleic anhydride and succinic anhydride.

Preferably, it is possible to use adipic acid, phthalic anhydride and/or isophthalic acid, and better still isophthalic acid alone.

There may also be mentioned the polycarboxylic acids chosen from, alone or as a mixture:
(i) polycarboxylic acids having a linear or branched, saturated or unsaturated chain, comprising at least one heteroatom chosen from O, N and/or S, in particular 1 to 10 heteroatoms which are identical or different, and/or comprising at least one perfluorinated radical —CF₂— or —CF₃ and having moreover at least 2 carboxyl groups COOH, in particular 2 to 4 COOH groups; and/or a cyclic anhydride of such a polycarboxylic acid;
and/or
(ii) saturated or unsaturated, or even aromatic, heterocyclic polycarboxylic acids comprising at least one heteroatom chosen from O, N and/or S, in particular 1 to 10, or even 1 to 4, heteroatoms which are identical or different, and at least 2 carboxyl groups COOH, in particular 2 to 4 COOH groups; and/or a cyclic anhydride of such a polycarboxylic acid;
and/or
(iii) polycarboxylic acids derived from sugar, which are liable to be obtained in particular by oxidation of an aldose, and comprising at least 2 carboxyl groups COOH, in particular 2 or 3 COOH groups; and/or a cyclic anhydride of such a polycarboxylic acid;
and/or
(iv) itaconic anhydride and the 1,4-monoanhydride of 1,4,5,8-naphthalenetetracarboxylic acid;
and/or
(v) polycarboxylic (including heterocyclic) amino acids, that is to say polycarboxylic acids having a linear, branched and/or cyclic, saturated or unsaturated chain optionally comprising at least one heteroatom chosen from O, N and/or S, in particular 1 to 10 heteroatoms which are identical or different, and/or optionally comprising at least one perfluorinated radical —CF₂— or —CF₃; and additionally comprising at least one primary, secondary or tertiary amine functional group (in particular NR1R2 with R1 and R2, independently of each other, chosen from H and C1-C12 alkyl), in particular 1 to 3 amine functional groups, which are identical or different, and having moreover at least 2 carboxyl groups COOH, in particular 2 to 4 COOH groups; and/or a cyclic anhydride of such a polycarboxylic acid.

There may be mentioned most particularly, alone or as a mixture, the following dicarboxylic acids:

(i)
2,2′-[1,5-pentanediylbis(thio)]bis-acetic acid
6,6′-[(1,2-dioxo-1,2-ethanediyl)diimino]bis-hexanoic acid
2,2′-sulphinylbis-acetic acid
4,13-dioxo-3,5,12,14-tetraazahexadecanedioic acid poly(ethylene glycol) disuccinate, in particular having a mass of 250-600
poly(ethylene glycol)bis(carboxymethyl)ether, in particular having a mass of 250-600
poly[oxy(1,2-dicarboxy-1,2-ethanediyl)], in particular having a DP<10
8-[(carboxymethyl)amino]-8-oxooctanoic acid
2,2′-[methylenebis(sulphonyl)]bis-acetic acid
4,4′-(1,6-hexanediyldiimino)bis[4-oxo-butanoic] acid
4,9-dioxo-3,5,8,10-tetraazadodecanedioic acid
4-[(1-carboxyethyl)amino]-4-oxo-butanoic acid
6-[(3-carboxy-1-oxopropyl)amino]hexanoic acid
N,N′-(1,6-dioxo-1,6-hexanediyl)bis-glycine
N,N′-(1,6-dioxo-1,6-hexanediyl)bis-phenylalanine
N,N′-(1,3-dioxo-1,3-propanediyl)bis-glycine
4,4′-[(1,4-dioxo-1,4-butanediyl)diimino]bis-butanoic acid
4,4′-[(1,6-dioxo-1,6-hexanediyl)diimino]bis-butanoic acid
6,6′-[1,6-hexanediylbis(iminocarbonylimino)]bis-hexanoic acid
N-benzoyl-S-(carboxymethyl)-Cysteine
N,N′-(2,2,3,3-tetrafluoro-1,4-dioxo-1,4-butanediyl)bis-Glycine
N,N′-(2,2,3,3-tetrafluoro-1,4-dioxo-1,4-butanediyl)bis-Alanine
4,4′-[(2,2,3,3-tetrafluoro-1,4-dioxo-1,4-butanoic acid
N,N′-(1,5-dioxo-1,5-pentanediyl)bis-Glycine
N,N′-(1,9-dioxo-1,9-nonanediyl)bis-Glycine
N,N′-(1,10-dioxo-1,10-decanediyl)bis[N-methyl-Glycine
bis(3-carboxypropyl)ester of propanedioic acid
7,16-dioxo-6,8,15,17-tetraazadocosanedioic acid
N-benzoyl-N-(2-carboxyethyl)-Glycine
[2-[(2-carboxymethyl)amino]-2-oxoethyl]-benzenepropanoic acid
[2-[(2-carboxyethyl)amino]-2-oxoethyl]-Benzenepropanoic acid
   (ii)
4,7,9,12-tetraoxapentadecanedioic acid
2,3-pyridinedicarboxylic acid
4-pyranone-2,6-dicarboxylic acid
2,5-pyrazinedicarboxylic acid
2,5-pyridinedicarboxylic acid
2,3-benzofurandicarboxylic acid
7-oxabicyclo[2.2.1]heptane-2,3-dicarboxylic acid
3,4-pyridinedicarboxylic acid
2,4-pyridinedicarboxylic acid
3,5-pyridinedicarboxylic acid
2,6-pyridinedicarboxylic acid
1H-imidazole-4,5-dicarboxylic acid
2,3-quinolinedicarboxylic acid
6,6,7,7-tetrafluoro-3-oxabicyclo[3.2.0]heptane-2,4-dicarboxylic acid
2,6-pyrazinedicarboxylic acid
2,6-dimethyl-3,5-pyridinedicarboxylic acid
1-phenyl-1H-pyrazole-3,4-dicarboxylic acid
2,5-furandicarboxylic acid
3,4-furandicarboxylic acid
1,2,5-thiadiazole-3,4-dicarboxylic acid
1,4-dihydro-1,2,4,5-tetrazine-3,6-dicarboxylic acid
2,3-furandicarboxylic acid
3,4-thiophenedicarboxylic acid
1H-1,2,3-triazole-4,5-dicarboxylic acid
2-methylimidazole-4,5-dicarboxylic acid
2,4-quinolinedicarboxylic acid
naphtho[2,1-b]furan-1,2-dicarboxylic acid
3,4-quinolinedicarboxylic acid
7-oxabicyclo[2.2.1]hept-5-ene-2,3-dicarboxylic acid
2,3-quinoxalinedicarboxylic acid
1,4-piperazinedicarboxylic acid
2,5-dimethyl-3,4-furandicarboxylic acid
tetrahydro-2,5-thiophenedicarboxylic acid
4-phenyl-3,5-pyridinedicarboxylic acid
thieno[3,2-b]thiophene-2,5-dicarboxylic acid
3-methyl-2,4-thiophenedicarboxylic acid
naphthostyril-5,6-dicarboxylic acid
3-phenyl-2,4-quinolinedicarboxylic acid
3,4-dimethyl-2,5-dicarboxythiophene
3,4-diphenyl-2,5-thiophenedicarboxylic acid
2,5-diphenyl-3,4-furandicarboxylic acid
7-oxo-7H-benzimidazo[2,1-a]benz[de]isoquinoline-3,4-dicarboxylic acid
2,3-dihydro-1,3-dioxo-1H-benz[de]isoquinoline-6,7-dicarboxylic acid
3,4-bis(phenylmethoxy)-2,5-furandicarboxylic acid
4,4′-bibenzoic acid 2,2′-sulphone
2,7-diphenyl-m-anthrazoline-4,5-dicarboxylic acid
2,4-pyrimidinedicarboxylic acid
2-phenyl-4,5-thiazoledicarboxylic acid
6-phenyl-2,3-pyridinedicarboxylic acid
5,6-dimethyl-2,3-pyrazinedicarboxylic acid
3,7-dibenzothiophenedicarboxylic acid
9-oxo-9H-xanthene-1,7-dicarboxylic acid
2-(1,1-dimethylethyl)-H-imidazole-4,5-dicarboxylic acid
6,7-quinolinedicarboxylic acid
6-methyl-2,3-pyridinedicarboxylic acid
4,5-pyrimidinedicarboxylic acid
2-methyl-3,4-furandicarboxylic acid
1,2-indolizinedicarboxylic acid
2,8-dibenzothiophenedicarboxylic acid
3,6-pyridazinedicarboxylic acid
1,10-phenanthroline-2,9-dicarboxylic acid
1,4,5,6-tetrahydro-5,6-dioxo-2,3-pyrazine-dicarboxylic acid
3,4-dimethoxy-2,5-furandicarboxylic acid
2-ethyl-4,5-imidazoledicarboxylic acid
2-propyl-1H-imidazole-4,5-dicarboxylic acid
4-phenyl-2,5-pyridinedicarboxylic acid
4,5-pyridazinedicarboxylic acid
1,4,5,8-tetrahydro-1,4:5,8-diepoxynaphthalene-4-a,8a-dicarboxylic acid
5,5-dioxide-2,8-dibenzothiophenedicarboxylic acid
pyrazolo[1,5-a]pyridine-2,3-dicarboxylic acid
2,3-dihydro-1H-pyrrolizine-1,7-dicarboxylic acid
6-methyl-2,4,5-pyridinetricarboxylic acid
pyrrolo[2,1,5-cd]indolizine-5,6-dicarboxylic acid
3,4-bis(2,2,3,3,4,4,4-heptafluorobutyl)-1H-pyrrole-2,5-dicarboxylic acid
6,7,9,10,17,18,20,21-octahydrodibenzo-[b,k][1,4,7,10,13,16]hexaoxacyclooctadecine-2,14-dicarboxylic acid
6,7,9,10,17,18,20,21-octahydrodibenzo-[b,k][1,4,7,10,13,16]hexaoxacyclooctadecine-2,13-dicarboxylic acid
2-methyl-3,4-quinolinedicarboxylic acid
4,7-quinolinedicarboxylic acid
3,5-isoxazoledicarboxylic acid
2-(trifluoromethyl)-3,4-furandicarboxylic acid
5-(trifluoromethyl)-2,4-furandicarboxylic acid
6-methyl-2,4-quinolinedicarboxylic acid
5-oxo-1,2-pyrrolidinedicarboxylic acid
5-ethyl-2,3-pyridinedicarboxylic acid
1,2-dihydro-2-oxo-3,4-quinolinedicarboxylic acid
4,6-phenoxathiindicarboxylic acid
10,10-dioxide 1,9-phenoxathiindicarboxylic acid
3,4-dihydro-2H-1,4-thiazine-3,5-dicarboxylic acid
2,7-di(tert-butyl)-9,9-dimethyl-4,5-xanthene-dicarboxylic acid
6-methyl-2,3-quinoxalinedicarboxylic acid
3,7-quinolinedicarboxylic acid
2,5-quinolinedicarboxylic acid
2-methyl-6-phenyl-3,4-pyridinedicarboxylic acid
3,4-dimethylthieno[2,3-b]thiophene-2,5-dicarboxylic acid
3,4-dimethoxythiophene-2,5-dicarboxylic acid
5-methyl-3,4-isoxazoledicarboxylic acid
2,6-bis(aminocarbonyl)-3,5-pyridinedicarboxylic acid
3,5-bis(aminocarbonyl)-2,6-pyrazinedicarboxylic acid
2,3-pyridinedicarboxylic acid
6-(1,1-dimethylethyl)-2-ethyl-3,4-pyridine-dicarboxylic acid
3-methyl-5-phenyl-2,4-thiophenedicarboxylic acid
1,2-dihydro-2-oxo-6-phenyl-3,5-pyridinedicarboxylic acid
8-methyl-2,4-quinolinedicarboxylic acid
4-ethyl-2,6-dimethyl-3,5-pyridinedicarboxylic acid
5-(phenoxymethyl)-2,4-furandicarboxylic acid
5-(acetylamino)-3-methyl-2,4-thiophenedicarboxylic acid
2-(4-heptylphenyl)-4,8-quinolinedicarboxylic acid
2,8-bis(4-heptylphenyl)pyrido[3,2-g]quinoline-4,6-dicarboxylic acid
1,2,3,4,6,7,8,9-octahydro-2,8-dioxopyrido[3,2]-quinoline-3,7-dicarboxylic acid
2,8-dimethylpyrido[3,2-g]quinoline-3,7-dicarboxylic acid
5,6-quinolinedicarboxylic acid
6-ethyl-2-methylcinchomeronic acid
2-methyl-6-propylcinchomeronic acid
6-isopropyl-2-methylcinchomeronic acid
6-tert-butyl-2-methylcinchomeronic acid
1,4-dimethyl-7-oxabicyclo[2.2.1]heptane-2,3-dicarboxylic acid
1,2-dihydro-2-oxo-3,8-quinolinedicarboxylic acid
1,2-dihydro-2-oxo-3,6-quinolinedicarboxylic acid
1,2-dihydro-2-oxo-3,7-quinolinedicarboxylic acid
3,7-dimethyl-2,8-diphenylpyrido[3,2-g]quinoline-4,6-dicarboxylic acid
8-methyl-2,3-quinolinedicarboxylic acid
3-[[(1,1-dimethylethyl)amino]sulphonyl]-2,5-thiophenedicarboxylic acid
4-(acetylamino)-2,3-thiophenedicarboxylic acid
2,5-pyridinedicarboxylic acid
2,6-pyridinedicarboxylic acid
2,4-thiophenedicarboxylic acid
2,5-thiophenedicarboxylic acid
1,4-pyran-2,6-dicarboxylic acid
   (iii)
ribaric acid
glucaric acid
xylaric acid
arabinaric acid
mannaric acid
idaric acid
altraric acid
L-glucaric acid
L-arabinaric acid
allaric acid
galactaric acid
meso-tartaric acid
D-glucaric acid
L-idaric acid
hexaric acid
2,3-dihydroxybutanedioic acid
D-tartaric acid
DL-tartaric acid
D-glucaric acid
tartaric acid
tetrahydroxysuccinic acid
2-carboxy-2,3-dideoxy-D-manno-2-octulopyranosonic acid
methyl 3-deoxy-D-arabino-2-heptulopyranosaric acid
D-lyxo-2-heptulopyranosaric acid
2,6-anhydro-L-glycero-L-galactoheptaric acid
   (iv)
1,4,5,8-naphthalenetetracarboxylic acid 1,4-monoanhydride
itaconic anhydride
   (v)
1,4-dihydro-4-oxo-2,6-pyridinedicarboxylic acid
2,6-piperidinedicarboxylic acid
1H-pyrrole-3,4-dicarboxylic acid
4-amino-2,6-dicarboxylic acid
1-methyl-1H-pyrazole-3,4-dicarboxylic acid
2,3-piperidinedicarboxylic acid
1-methyl-1H-imidazole-4,5-dicarboxylic acid
2,4-thiazolidinedicarboxylic acid
1-(phenylmethyl)-1H-imidazole-4,5-dicarboxylic acid
5-amino-6-oxo-2,3-piperidinedicarboxylic acid
5-amino-6-oxo-2,4-piperidinedicarboxylic acid
5-amino-6-oxo-2,3-piperidinedicarboxylic acid
5-amino-6-oxo-[2S-(2α,4β,5α)]-2,4-piperidine-dicarboxylic acid
(2S,4R)-2,4-pyrrolidinedicarboxylic acid
(2S-cis)-2,4-pyrrolidinedicarboxylic acid
2-amino-1H-imidazole-4,5-dicarboxylic acid
2,5-pyrrolidinedicarboxylic acid
4-amino-3,5-isothiazoledicarboxylic acid
1-methyl-1H-pyrazole-3,5-dicarboxylic acid
7-(diethylamino)-2-oxo-2H-1-benzopyran-3,4-dicarboxylic acid
3,4-diethyl-1H-pyrrole-2,5-dicarboxylic acid
1-phenyl-1H-pyrrole-3,4-dicarboxylic acid
cis-2,3-piperazinedicarboxylic acid
2,3-piperazinedicarboxylic acid
2,5-piperazinedicarboxylic acid
2,6-piperazinedicarboxylic acid
2-amino-3,5-pyridinedicarboxylic acid
2-methylpyrrole-3,4-dicarboxylic acid
4-(methylamino)-2,6-pyridinedicarboxylic acid
2-amino-6-methyl-3,4-pyridinedicarboxylic acid
5-amino-2-methyl-3,4-pyridinedicarboxylic acid
2-amino-6-methyl-3,5-pyridinedicarboxylic acid
2,5-dimethylpyrrole-3,4-dicarboxylic acid
2-amino-6-hydroxy-3,5-pyridinedicarboxylic acid
2,4-pyrrolidinedicarboxylic acid
1H-indole-2,4-dicarboxylic acid
1H-indole-2,6-dicarboxylic acid
1H-indole-2,5-dicarboxylic acid
5-phenyl-2,4-pyrrolidinedicarboxylic acid
5-methyl-2,4-pyrrolidinedicarboxylic acid
trans-2,4-azetidinedicarboxylic acid
cis-2,4-azetidinedicarboxylic acid
3,5-piperidinedicarboxylic acid
2,3-pyrrolidinedicarboxylic acid
2,3-azetidinedicarboxylic acid
3,4-pyrrolidinedicarboxylic acid
2,3-dihydro-6H-1,4-dioxino[2,3-c]pyrrole-5,7-dicarboxylic acid
1H-imidazole-2,4-dicarboxylic acid
1-butyl-1H-pyrrole-2,3-dicarboxylic acid
3-amino-1-oxide-2,4-pyridinedicarboxylic acid
2,3-dihydro-5-phenyl-1H-pyrrolizine-6,7-dicarboxylic acid
3a,4,5,9b-tetrahydro-3H-cyclopenta[c]quinoline-4,6-dicarboxylic acid
3a,4,5,9b-tetrahydro-3H-cyclopenta[c]quinoline-4,8-dicarboxylic acid
2,3-dihydro-1H-imidazole-4,5-dicarboxylic acid
5-amino-6-methyllutidinic acid
1H-indole-3,7-dicarboxylic acid
3,3-dimethyl-2,6-piperidinedicarboxylic acid
1-butyl-2,5-pyrrolidinedicarboxylic acid
1H-indole-4,6-dicarboxylic acid
1-(phenylmethyl)-3,4-pyrrolidinedicarboxylic acid
3-(carboxymethyl)-1H-indole-2,6-dicarboxylic acid
3,4-bis(2,2,2-trifluoroethyl)-1H-pyrrole-2,5-dicarboxylic acid
9-hexyl-9H-carbazole-3,6-dicarboxylic acid
3-methyl-5-(1-piperazinylsulphonyl)-2,4-thiophene-dicarboxylic acid
2,3,4,9-tetrahydro-1H-carbazole-5,7-dicarboxylic acid
2,3-dimethyl-1H-indole-4,6-dicarboxylic acid
7-amino-1,4-dihydro-4-oxo-3,6-quinolinedicarboxylic acid
5-amino-3-methyl-2,4-thiophenedicarboxylic acid
(m-tolylimino)diacetic acid
(o-tolylimino)diacetic acid
D-cystathionine
phenethyliminodiacetic acid
2-benzyl-2,2′-iminodiacetic acid
L-α-glutamyl-L-alanyl-L-alanine
N,N′-dibenzylethylenediaminediacetic acid
N-L-γ-glutamyl-D-alanine
glycyl-L-glutamylglycine
N-(carboxymethyl)-N-(tetrahydro-1,1-dioxido-3-thienyl)glycine
N-(2-carboxyethyl)-N-phenyl-β-alanine
N-(carboxymethyl)-N-octylglycine
N-(tert-butoxycarbonyl)iminodiacetic acid
N-(carboxymethyl)-L-alanine
N-(6-aminohexyl)-N-(carboxymethyl)glycine
N-(carboxymethyl)-N-tetradecylglycine
N-(1-carboxyethyl)-D-alanine
N-(carboxymethyl)-D-alanine
decyliminodiacetic acid
3,3′-(dimethylhydrazono)bispropanoic acid
N-(carboxymethyl)-N-[2-(2,6-dioxo-4-morpholinyl)-ethyl]glycine
N-α-aspartylglycine
N-β-aspartylglycine
N-L-α-aspartyl-β-alanine
3,4-xylylamino-N,N-diacetic acid
N-(1-carboxyethyl)alanine
N-(carboxymethyl)alanine
N,N′-methylenebisglycine
N-(aminomethyl)-N-(carboxymethyl)glycine
2,2′-(methylhydrazono)bisacetic acid
N-(2-carboxyethyl)-N-(4-methylphenyl)-β-alanine
N-(2-carboxyethyl)-N-(3-methylphenyl)-β-alanine
3-[(carboxymethyl)amino]alanine
D-α-aspartyl-D-alanine
N-(2-carboxyethyl)-N-(1-oxohexadecyl)-β-alanine
N-(2-carboxyethyl)-N-(1-oxodecyl)-β-alanine
N-(2-carboxyethyl)-N-(1-oxotetradecyl)-β-alanine
amino[(carboxymethyl)thio]acetic acid
N,N′-1,6-hexanediylbis-β-alanine
N-(carboxymethyl)-N-phenyl-β-alanine
N-(1-carboxyethyl)-L-alanine
L-glutamic acid
L-aspartic acid

There may also be mentioned, alone or as a mixture, the following tricarboxylic and tetracarboxylic acids, and their anhydrides:
3,3′,3″-[1,2,3-propanetriyltris(oxy)tris-propanoic acid
pyrazinetricarboxylic acid
4-(3-carboxyphenyl)-2,5-pyridinedicarboxylic acid
3-(carboxymethyl)-2,4-quinolinedicarboxylic acid
3-(carboxymethyl)-1H-indole-2,5-dicarboxylic acid
3-C-carboxy-2-deoxy-D-threopentaric acid
hydroxycitric acid
D-glucopyranuronosyl-D-arabino-2-hexylofuranosidaric acid
2,3,5,6-pyridinetetracarboxylic acid
N,N′-1,2-ethanediylbis[N-(carboxymethyl)-β-alanine
L-α-aspartyl-L-aspartic acid
4-[bis(carboxymethyl)amino]benzoic acid
7-[bis(carboxymethyl)amino]heptanoic acid
N-(2-carboxyethyl)aspartic acid
3-[bis(2-carboxyethyl)amino]benzoic acid
4-[bis(2-carboxyethyl)amino]benzoic acid.

Preferably, it is possible to use 6,6′-[(1,2-dioxo-1,2-ethanediyl)diimino]bis-hexanoic acid, 2,2′-sulphinylbis-acetic acid, 4,13-dioxo-3,5,12,14-tetra-azahexadecanedioic acid, poly(ethylene glycol)-disuccinate, poly(ethylene glycol)bis(carboxymethyl)-ether, 8-[(carboxymethyl)amino]-8-oxo-octanoic acid, 2,2′-[methylenebis(sulphonyl)]bis-acetic acid, 4,4′-(1,6-hexanediyldiimino)bis[4-oxo-butanoic] acid, 4,9-dioxo-3,5,8,10-tetraazadodecanedioic acid, 4-[(1-carboxyethyl)amino]-4-oxo-butanoic acid, 6-[(3-carboxy-1-oxopropyl)amino]hexanoic acid, N,N′-(1,6-dioxo-1,6-hexanediyl)bis-glycine, N,N′-(1,3-dioxo-1,3-propane-diyl)bis-glycine, 4,7,9,12-tetraoxapentadecanedioic acid, 4-pyranone-2,6-dicarboxylic acid, 2,5-pyrazine-dicarboxylic acid, 1H-imidazole-4,5-dicarboxylic acid, 2,6-pyrazinedicarboxylic acid, 2,5-furandicarboxylic acid, 3,4-furandicarboxylic acid, 2,3-furandicarboxylic acid, 2,5-diphenyl-3,4-furandicarboxylic acid, 2-methyl-3,4-furandicarboxylic acid, D-tartaric acid, DL-tartaric acid, L-tartaric acid, galactaric acid, D-glucaric acid, 2,5-pyridinedicarboxylic acid, 2,5-pyrrolidinedicarboxylic acid, 1-phenyl-1H-pyrrole-3,4-dicarboxylic acid, 2,4-pyrrolidinedicarboxylic acid, 5-phenyl-2,4-pyrrolidinedicarboxylic acid, 3,5-piperidinedicarboxylic acid, 3,4-pyrrolidine-dicarboxylic acid, 1-butyl-2,5-pyrrolidinedicarboxylic acid, 1-(phenylmethyl)-3,4-pyrrolidinedicarboxylic acid, N-(2-carboxyethyl)-N-phenyl-β-Alanine, N-(carboxymethyl)-N-octyl-glycine, N-(1-carboxyethyl)-L-Alanine, L-glutamic acid, L-aspartic acid, N-(2-carboxyethyl)-Aspartic acid; and mixtures thereof.

Most particularly preferred are 2,2′-sulphinylbis-acetic acid, 2,2′-[methylenebis(sulphonyl)]bis-acetic acid, N,N′-(1,3-dioxo-1,3-propanediyl)bis-glycine, 2,5-furandicarboxylic acid, D-tartaric acid, DL-tartaric acid, L-tartaric acid, galactaric acid, L-glutamic acid, L-aspartic acid, and mixtures thereof.

It is also possible to use a lactone comprising at least one carboxyl group, in particular 1, 2 or 3 COOH groups. Preferably, the lactones comprise 5 to 14 carbon atoms, in particular 6 to 13, or even 6 to 12 carbon atoms.

The following lactones may be most particularly mentioned, alone or as a mixture:
tetrahydro-2,2-dimethyl-5-oxo-3-furancarboxylic acid
4,7,7-trimethyl-3-oxo-2-oxabicyclo[2.2.1]heptane-1-carboxylic acid
4,6-dimethyl-2-oxo-2H-pyran-5-carboxylic acid
2-oxo-2H-pyran-5-carboxy-2-pentenedioic acid
2-oxo-2H-1-benzopyran-3-carboxylic acid
2-oxo-2H-pyran-6-carboxylic acid
1,3-dihydro-3-oxo-1-isobenzofurancarboxylic acid
4-methyl-2-oxo-2H-1-benzopyran-3-carboxylic acid
1-oxo-1H-2-benzopyran-3-carboxylic acid
8-methoxy-2-oxo-2H-1-benzopyran-3-carboxylic acid
2-oxo-1-oxaspiro[4.5]decane-4-carboxylic acid
2-oxo-2H-pyran-3-carboxylic acid
4-methyl-2-oxo-2H-pyran-6-carboxylic acid
3-oxo-3H-naphtho[2,1-b]pyran-2-carboxylic acid
tetrahydro-5-oxo-2,3-furandicarboxylic acid
1,3-dihydro-3-oxo-4-isobenzofurancarboxylic acid
1,3-dihydro-1-oxo-5-isobenzofurancarboxylic acid
hexahydro-2-oxo-3,5-methano-2H-cyclopenta[b]furan-7-carboxylic acid
6-methyl-2,4-dioxo-2H-pyran-5-carboxylic acid
1-oxo-3-isochromancarboxylic acid
2-oxo-2H-1-benzopyran-6-carboxylic acid
6-methyl-2-oxo-2H-1-benzopyran-3-carboxylic acid
2,5-dihydro-4,5,5-trimethyl-2-oxo-3-furancarboxylic acid
tetrahydro-5-oxo-2-phenyl-3-furancarboxylic acid tetrahydro-5-oxo-4-propyl-2-furoic acid
2-butyl-2,3-dideoxypentaric acid
2-oxo-2H-1-benzopyran-7-carboxylic acid
2-oxo-1-oxaspiro[4.4]nonane-4-carboxylic acid
4-ethyltetrahydro-5-oxo-2-furoic acid
5-ethyltetrahydro-2,3-dimethyl-6-oxo-2H-pyran-2-carboxylic acid
7-methoxy-2-oxo-2H-1-benzopyran-3-carboxylic acid
2-oxo-2H-1-benzopyran-4-carboxylic acid
2-oxo-6-pentyl-2H-pyran-3-carboxylic acid
7-oxo-4-oxepanecarboxylic acid
3-(carboxymethyl)-2,3-dideoxypentaric acid
2,3-dihydro-2-oxo-7-benzofurancarboxylic acid
1,3,4,5-tetrahydro-1-oxo-2-benzoxepin-7-carboxylic acid
3,4-dihydro-3-oxo-1H-2-benzopyran-6-carboxylic acid
2,3,4,5-tetrahydro-2-oxo-1-benzoxepin-7-carboxylic acid
3,4-dihydro-1-oxo-1H-2-benzopyran-8-carboxylic acid
1,3,4,5-tetrahydro-3-oxo-2-benzoxepin-9-carboxylic acid
1,3,4,5-tetrahydro-3-oxo-2-benzoxepin-7-carboxylic acid
3,4-dihydro-2-oxo-2H-1-benzopyran-8-carboxylic acid
1,3,4,5-tetrahydro-1-oxo-2-benzoxepin-9-carboxylic acid
3,4-dihydro-1-oxo-1H-2-benzopyran-6-carboxylic acid
3,4-dihydro-3-oxo-1H-2-benzopyran-8-carboxylic acid
2,3,4,5-tetrahydro-2-oxo-1-benzoxepin-9-carboxylic acid
isocitric acid lactone
5-oxo-2-tetrahydrofurancarboxylic acid.

Preferably, it is possible to use tetrahydro-5-oxo-2,3-furandicarboxylic acid, 1,3-dihydro-3-oxo-4-isobenzofurancarboxylic acid, 1,3-dihydro-1-oxo-5-isobenzofurancarboxylic acid, tetrahydro-5-oxo-2-phenyl-3-furancarboxylic acid, isocitric acid lactone, 5-oxo-2-tetrahydrofurancarboxylic acid and mixtures thereof.

The polycarboxylic acid and/or its cyclic anhydride and/or the lactone, and mixtures thereof, preferably represents 1 to 40% by weight, or even 5 to 35% by weight, in particular 10 to 30% by weight, and even better 14 to 25% by weight, of the total weight of the final polycondensate.

In a preferred embodiment of the invention, the aromatic monocarboxylic acid is present in a molar quantity of less than or equal to that of the non-aromatic monocarboxylic acids (of high and low melting points); in particular the ratio between the number of moles of aromatic monocarboxylic acid and the total number of moles of non-aromatic monocarboxylic acids is preferably between 0.08 and 0.70, in particular between 0.10 and 0.60, in particular between 0.12 and 0.40.

It is observed that this makes it possible in particular to obtain a polymer that is advantageously soluble in the oily media generally used to formulate cosmetic compositions of the lipstick or foundation type; moreover, the film obtained has sufficient rigidity and flexibility for its use in this type of formulation, while having a gloss and a gloss staying power as desired.

Preferably, the polycondensate according to the invention can be obtained by the reaction:
of at least one polyol chosen from, alone or as a mixture, 1,2,6-hexanetriol, trimethylolethane, trimethylolpropane, glycerol; pentaerythritol, erythritol, diglycerol, ditrimethylolpropane; xylitol, sorbitol, mannitol, dipentaerythritol and/or triglycerol;
   preferably present in a quantity of 10 to 30% by weight, in particular 12 to 25% by weight, and even better 14 to 22% by weight, relative to the total weight of the final polycondensate;
of at least one non-aromatic monocarboxylic acid having a melting point greater than or equal to 25° C., chosen from, alone or as a mixture, decanoic acid, lauric acid, tridecanoic acid, myristic acid, palmitic acid, stearic acid, arachidic acid, behenic acid, cerotic acid, petroselinic acid, vaccenic acid, elaidic acid, gondoic acid, gadoleic acid, erucic acid, nervonic acid;
   preferably present in a quantity of 22 to 80% by weight, in particular 25 to 75% by weight, and even better 27 to 70% by weight, relative to the total weight of the final polycondensate;
of at least one non-aromatic monocarboxylic acid having a melting point of less than 25° C., chosen from, alone or as a mixture, caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, isononanoic acid, isostearic acid; caproleic acid, obtusilic acid, undecylenic acid, dodecylenic acid, linderic acid, myristoleic acid, physeteric acid, tsuzuic acid, palmitoleic acid, oleic acid, linoleic acid, linolenic acid, arachidonic acid;
   preferably present in a quantity of 0.1 to 35% by weight, in particular 0.5 to 32% by weight, and even better 1 to 30% by weight, relative to the total weight of the final polycondensate;
of at least one aromatic monocarboxylic acid chosen from, alone or as a mixture, benzoic acid, o-toluic acid, m-toluic acid, p-toluic acid, 1-naphthoic acid, 2-naphthoic acid, 4-tert-butylbenzoic acid, 1-methyl-2-naphthoic acid, 2-isopropyl-1-naphthoic acid; preferably present in a quantity of 0.1 to 10% by weight, in particular 1 to 9.5% by weight, or even 1.5 to 8% by weight, relative to the total weight of the final polycondensate; and
of at least one polycarboxylic acid or one of its anhydrides, chosen from, alone or as a mixture, decanedioic acid, dodecanedioic acid, cyclopropanedicarboxylic acid, cyclohexanedicarboxylic acid, cyclobutanedicarboxylic acid, 1,4-naphthalenedicarboxylic acid, 2,3-naphthalenedicarboxylic acid, 2,6-naphthalenedicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid; cyclohexanetricarboxylic acid, trimellitic acid, 1,2,3-benzenetricarboxylic acid, 1,3,5-benzenetricarboxylic acid, butanetetracarboxylic acid, pyromellitic acid, phthalic anhydride, trimellitic anhydride, maleic anhydride, succinic anhydride, 2,2′-sulphinylbis-acetic acid, 2,2′-[methylenebis(sulphonyl)]bis-acetic acid, N,N′-(1,3-dioxo-1,3-propanediyl)bis-glycine, 2,5-furandicarboxylic acid, D-tartaric acid, DL-tartaric acid, L-tartaric acid, galactaric acid and mixtures thereof; preferably present in a quantity of 1 to 40% by weight, in particular 10 to 30% by weight, and even better 14 to 25% by weight, relative to the total weight of the final polycondensate.

Preferably, the polycondensate according to the invention can be obtained by the reaction:
of at least one polyol chosen from, alone or as a mixture, glycerol, pentaerythritol, sorbitol and mixtures thereof, and better still pentaerythritol alone; present in a quantity of 10 to 30% by weight, in particular 12 to 25% by weight, and even better 14 to 22% by weight, relative to the total weight of the final polycondensate;
of at least one non-aromatic monocarboxylic acid chosen from, alone or as a mixture, lauric acid, palmitic acid, stearic acid, behenic acid and mixtures thereof, and better still stearic acid alone; preferably present in a quantity of 22 to 80% by weight, in particular 25 to 75% by weight, and even better 27 to 70% by weight, relative to the total weight of the final polycondensate;
of at least one non-aromatic monocarboxylic acid chosen from, alone or as a mixture, isooctanoic acid, isononanoic acid, isostearic acid; preferably present in a quantity of 0.1 to 35% by weight, in particular 0.5 to 32% by weight, and even better 1 to 30% by weight, relative to the total weight of the final polycondensate;
of at least one aromatic monocarboxylic acid chosen from, alone or as a mixture, benzoic acid, o-toluic acid, m-toluic acid, 1-naphthoic acid, and better still benzoic acid alone; present in a quantity of 0.1 to 10% by weight, in particular 1 to 9.5% by weight, or even 1.5 to 8% by weight relative to the total weight of the final polycondensate; and
of at least one polycarboxylic acid or one of its anhydrides, chosen from, alone or as a mixture, phthalic anhydride and isophthalic acid, and better still isophthalic acid alone; present in a quantity of 1 to 40% by weight, in particular 10 to 30% by weight, and even better 14 to 25% by weight, relative to the total weight of the final polycondensate.

In a preferred embodiment of the invention, the polycondensate can be prepared exclusively (or solely) from the monomers/categories of monomers mentioned in the present description, that is to say that it does not comprise other additional monomers/categories of monomers; in this case, the polycondensate is therefore constituted exclusively (or solely) of 10-30% by weight of polyol comprising 3 to 6 hydroxyl groups; from 22-80% by weight of linear, branched and/or cyclic, saturated or unsaturated, non-aromatic monocarboxylic acid comprising 10-32 carbon atoms, and having a melting point greater than or equal to 25° C.; from 0.1 to 35% by weight, of linear, branched and/or cyclic, saturated or unsaturated, non-aromatic monocarboxylic acid comprising 6 to 32 carbon atoms, and having a melting point strictly less than 25° C.; from 0.1 to 10% by weight of aromatic monocarboxylic acid comprising 7-11 carbon atoms, optionally substituted with 1-3 linear, branched and/or cyclic, saturated or unsaturated alkyl radicals which comprise 1-32 carbon atoms; from 1 to 40% by weight of linear, branched and/or cyclic, saturated or unsaturated, or even aromatic, polycarboxylic acid comprising at least 2 carboxyl groups COOH which may comprise heteroatoms; and/or a cyclic anhydride of such a polycarboxylic acid; and/or a lactone comprising at least one COOH group.

Preferably, the polycondensate according to the invention has:
an acid value, expressed in mg of potassium hydroxide per g of polycondensate, greater than or equal to 1; in particular between 2 and 30, and better still between 2.5 and 15; and/or
a hydroxyl value, expressed in mg of potassium hydroxide per g of polycondensate, greater than or equal to 30; in particular between 30 and 120, and better still between 40 and 90.

These acid and hydroxyl values can be easily determined by persons skilled in the art by the usual analytical methods.

Preferably, the polycondensate according to the invention has a viscosity, measured at 110° C., between 20 and 4000 mPa·s, in particular between 30 and 3500 mPa·s, or even between 40 and 3000 mPa·s, and better still between 50 and 2500 mPa·s. This viscosity is measured in the manner described before the example.

Preferably, the polycondensate according to the invention has a weight-average molecular mass (Mw) between 1500 and 300 000, or even between 2000 and 200 000, and in particular between 3000 and 100 000.

The average molecular weight may be determined by gel permeation chromatography or by light scattering, depending on the solubility of the polymer considered.

Moreover, the polycondensate is advantageously soluble in the cosmetic oily media customarily used, and in particular in vegetable oils, alkanes, fatty esters, fatty alcohols, silicone oils, and more particularly in media comprising isododecane, Parleam, isononyl isononanoate, octyldodecanol, phenyl trimethicone and/or C12-C15 alkyl benzoate.

The expression soluble is understood to mean that the polymer forms a clear solution in at least one solvent chosen from isododecane, Parleam, isononyl isononanoate, octyldodecanol and C12-C15 alkyl benzoate, in an amount of at least 50% by weight, at 70° C. Some compounds even have a particularly advantageous solubility in certain fields of application, namely a solubility in at least one of the solvents mentioned above, in an amount of at least 50% by weight at 25° C.

The polycondensate according to the invention may be prepared by the esterification/polycondensation methods customarily used by persons skilled in the art. By way of illustration, a general method of preparation comprises:
in mixing the polyol and the aromatic and non-aromatic monocarboxylic acids,
in heating the mixture under an inert atmosphere, first up to the melting point (generally 100-130° C.) and then at a temperature between 150 and 220° C. until there is complete consumption of the monocarboxylic acids (obtained when the acid value is less than or equal to 1), preferably while continuously distilling the water formed, and then
in optionally cooling the mixture to a temperature of between 90 and 150° C.,
in adding the polycarboxylic acid and/or the cyclic anhydride and/or the lactone, all at once or sequentially, and then
in again heating to a temperature less than or equal to 220° C., in particular between 170 and 220° C., preferably while continuing to remove the water formed, until the desired characteristics in terms of acid value, viscosity, hydroxyl value and solubility are obtained.

It is possible to add conventional esterification catalysts, for example of the sulphonic acid type (in particular at a concentration by weight between 1 and 10%) or of the titanate type (in particular at a concentration by weight between 5 and 100 ppm).

It is also possible to carry out the reaction, entirely or partially, in an inert solvent such as xylene and/or under reduced pressure, in order to facilitate the removal of water.

Advantageously, neither catalyst nor solvent is used.

The method of preparation may additionally comprise a step of adding at least one antioxidant to the reaction medium, in particular at a concentration by weight between 0.01 and 1%, relative to the total weight of monomers, so as to limit the possible degradations linked to prolonged heating.

The antioxidant may be of the primary type or of the secondary type, and may be chosen from hindered phenols, aromatic secondary amines, organophosphate compounds, sulphur compounds, lactones, acrylated bisphenols; and mixtures thereof.

Among the antioxidants particularly preferred, there may be mentioned in particular BHT, BHA, TBHQ, 1,3,5-trimethyl-2,4,6-tris(3,5-di-tert-butyl-4-hydroxybenzyl)benzene, octadecyl 3,5-di-tert-butyl-4-hydroxycinnamate, tetrakis-methylene-3-(3,5-di-tert-butyl-4-hydroxyphenyl)propionate methane, octadecyl 3-(3,5-di-tert-butyl-4-hydroxyphenyl)propionate, 2,5-di-tert-butylhydroquinone, 2,2-methybis(4-methyl-6-tert-butylphenol), 2,2-methylenebis(4-ethyl-6-tert-butyl-phenol), 4,4-butylidenebis(6-tert-butyl-m-cresol), N,N-hexamethylene bis(3,5-di-tert-butyl-4-hydroxyhydrocinnamamide), pentaerythritol tetrakis(3-(3,5-di-tert-butyl-4-hydroxyphenyl)propionate), in particular that marketed by CIBA under the name IRGANOX 1010; octadecyl 3-(3,5-di-tert-butyl-4-hydroxyphenyl)propionate, in particular that marketed by CIBA under the name IRGANOX 1076; 1,3,5-tris(3,5-di-tert-butyl-4-hydroxybenzyl)-1,3,5-triazine-2,4,6-(1H,3H,5H)trione, in particular that marketed by Mayzo of Norcross, Ga. under the name BNX 3114; distearyl pentaerythritol diphosphite, tris(2,4-di-tert-butylphenyl)phosphite, in particular that marketed by CIBA under the name IRGAFOS 168; dilauryl thiodipropionate, in particular that marketed by CIBA under the name IRGANOX PS800; bis(2,4-di-tert-butyl)pentaerythritol diphosphite, in particular that marketed by CIBA under the IRGAFOS 126; bis(2,4-bis[2-phenylpropan-2-yl]phenyl)pentaerythritol diphosphite, triphenylphosphite, (2,4-di-tert-butylphenyl)pentaerythritol diphosphite, in particular that marketed by GE Speciality Chemicals under the name ULTRANOX 626; tris(nonylphenyl)phosphite, in particular that marketed by CIBA under the name IRGAFOS TNPP; 1:1 mixture of N,N-hexamethylenebis(3,5-di-tert-butyl-4-hydroxyhydrocinnamamide) and of tris(2,4-di-tert-butylphenyl)phosphate, in particular that marketed by CIBA under the name Irganox B 1171; tetrakis(2,4-di-tert-butylphenyl)phosphite, in particular that marketed by CIBA under the name IRGAFOS P-EPQ; distearyl thiodipropionate, in particular that marketed by CIBA under the name IRGANOX PS802; 2,4-bis(octylthiomethyl)-o-cresol, in particular that marketed by CIBA under the name IRGANOX 1520; 4,6-bis(dodecylthiomethyl)-o-cresol, in particular that marketed by CIBA under the name IRGANOX 1726.

The polycondensates according to the invention may be used very advantageously in a composition, in particular a cosmetic or pharmaceutical composition, which comprises, moreover, a physiologically, in particular cosmetically or pharmaceutically, acceptable medium, that is to say a medium that is compatible with the cutaneous tissues such as the skin of the face or of the body, and keratin materials such as the hair, the eyelashes, the eyebrows of the nails.

The quantity of polycondensate present in the compositions of course depends on the type of composition and on the desired properties and may vary within a very broad range, generally between 0.1 and 70% by weight, preferably between 1 and 50% by weight, in particular between 10 and 45% by weight, or even between 20 and 40% by weight, and even better between 25 and 35% by weight, relative to the weight of the final cosmetic or pharmaceutical composition.

The composition may then comprise, depending on the application envisaged, the usual constituents for this type of composition.

The composition according to the invention may advantageously comprise a liquid fatty phase, which may constitute a solvent medium for the polymers according to the invention, and which may comprise at least one compound chosen from volatile or non-volatile, carbonaceous, hydrocarbon, fluorinated and/or silicone oils and/or solvents of inorganic, animal, plant or synthetic origin, alone or as a mixture insofar as they form a homogeneous and stable mixture and are compatible with the use envisaged.

The expression “volatile” is understood to mean, for the purposes of the invention, any compound liable to evaporate on contact with keratin materials, or the lips, in less than one hour, at room temperature (25° C.) and atmospheric pressure (1 atm). In particular, this volatile compound has a non-zero vapour pressure, at room temperature and atmospheric pressure, in particular ranging from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg). By contrast, the expression “non-volatile” is understood to mean a compound which remains on the keratin materials or the lips at room temperature and atmospheric pressure for at least one hour and which has in particular a vapour pressure of less than 10⁻³ mmHg (0.13 Pa).

Preferably, the physiologically acceptable medium of the composition according to the invention may comprise, in a liquid fatty phase, at least one oil and/or solvent which may be chosen from, alone or as a mixture:

1/ Esters of monocarboxylic acids with monoalcohols and polyalcohols; advantageously, the ester is a C12-C15 alkyl benzoate or corresponds to the following formula: R′₁—COO—R′₂ where:

R′₁ represents a linear or branched alkyl radical of 1 to 40 carbon atoms, preferably of 7 to 19 carbon atoms, optionally comprising one or more ethylenic double bonds, optionally substituted and whose hydrocarbon chain may be interrupted by one or more heteroatoms chosen from N and O and/or one or more carbonyl functional groups, and
R′₂ represents a linear or branched alkyl radical of 1 to 40 carbon atoms, preferably of 3 to 30 carbon atoms and even better of 3 to 20 carbon atoms, optionally comprising one or more ethylenic double bonds, optionally substituted and whose hydrocarbon chain may be interrupted by one or more heteroatoms chosen from N and C and/or one or more carbonyl functional groups.

The expression “optionally substituted” is understood to mean that R′₁ and/or R′₂ may carry one or more substituents chosen, for example, from the groups comprising one or more heteroatoms chosen from C and/or N, such as amino, amine, alkoxy, hydroxyl.

Examples of R′₁ groups are those derived from fatty acids, preferably higher fatty acids, chosen from the group constituted of acetic, propionic, butyric, caproic, caprylic, pelargonic, capric, undecanoic, lauric, myristic, palmitic, stearic, isostearic, arachidic, behenic, oleic, linolenic, linoleic, oleostearic, arachidonic and erucic acids, and mixtures thereof. Preferably, R′₁ is an unsubstituted branched alkyl group of 4 to 14 carbon atoms, preferably of 8 to 10 carbon atoms and R₂ is an unsubstituted branched alkyl group of 5 to 15 carbon atoms, preferably of 9 to 11 carbon atoms.

There may preferably be mentioned in particular C₈-C₄₈ esters, optionally incorporating in their hydrocarbon chain one or more heteroatoms among N and O and/or one or more carbonyl functional groups; and more particularly purcellin oil (cetostearyl octanoate), isononyl isononanoate, isopropyl myristate, isopropyl palmitate, 2-ethylhexyl palmitate, 2-octyldodecyl stearate, 2-octyldodecyl erucate, isostearyl isostearate, C₁₂ to C₁₅ alcohol benzoate, hexyl laurate, diisopropyl adipate; and alcohol or polyalcohol heptanoates, octanoates, decanoates or ricinoleates, for example fatty alcohols such as propylene glycol dioctanoate, and isopropyl N-lauroyl sarcosinate (in particular Eldew-205SL from Ajinomoto); hydroxyl esters such as isostearyl lactate, diisostearyl malate; and pentaerythritol esters; branched C8-C16 esters, in particular isohexyl neopentanoate.

2/ Plant hydrocarbon oils having a high content of triglycerides constituted of fatty acid esters of glycerol in which the fatty acids may have varied chain lengths from C₄ to C₂₄, it being possible for these chains to be linear or branched, saturated or unsaturated; these oils are in particular wheatgerm oil, corn oil, sunflower oil, shea oil, castor oil, sweet almond oil, macadamia oil, apricot oil, soybean oil, rapeseed oil, cotton seed oil, lucern oil, poppy seed oil, pumpkin seed oil, sesame oil, gourd oil, avocado oil, hazelnut oil, grape seed or blackcurrant seed oil, evening primrose oil, millet oil, barley oil, quinoa oil, olive oil, rye oil, safflower oil, candlenut oil, passion flower oil, musk rose oil, jojoba oil, palm oil, calophyllum oil; or alternatively triglycerides of caprylic/capric acids such as those sold by the company Stearinerie Dubois or those sold under the names “Miglyol 810®”, “812®” and “818®” by the company Dynamit Nobel.

3/ C6-C32, in particular C12-C26, alcohols, and in particular monoalcohols, such as oleyl alcohol, linoleyl alcohol, linolenyl alcohol, isostearyl alcohol, 2-hexyldecanol, 2-butyloctanol, 2-undecylpentadecanol and octyldodecanol.

4/ Volatile on non-volatile, linear or branched hydrocarbon oils of synthetic or mineral origin, which may be chosen from hydrocarbon oils having from 5 to 100 carbon atoms, and in particular petroleum jelly, polydecenes, hydrogenated polyisobutenes such as Parleam, squalane, perhydrosqualene and mixtures thereof.

There may be mentioned more particularly linear, branched and/or cyclic C5-C48 alkanes, and preferably branched C8-C16 alkanes such as C8-C16 isoalkanes of petroleum origin (also called isoparaffins); in particular decane, heptane, dodecane, cyclohexane; and isododecane, isodecane, isohexadecane.

5/ Volatile or non-volatile silicone oils;

as volatile silicone oils, there may be mentioned linear or cyclic volatile silicone oils, in particular those having a viscosity of less than 8 centistokes, and having in particular from 2 to 10 silicon atoms, these silicones optionally containing alkyl or alkoxy groups having from 1 to 22 carbon atoms; and in particular octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, methylhexyldimethylsiloxane and mixtures thereof.

The non-volatile silicone oils which can be used according to the invention may be polydimethylsiloxanes (PDMS), polydimethylsiloxanes containing alkyl or alkoxy groups which are pendent and/or at the silicone chain end, groups each having from 2 to 24 carbon atoms, phenylated silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates.

Preferably, the physiologically acceptable medium of the composition according to the invention comprises, in a liquid fatty phase, at least one oil and/or solvent chosen from, alone or as a mixture, isododecane, Parleam, isononyl isononanoate, octyldodecanol, phenyl trimethicone, C12-C15 alkyl benzoates and/or D5 (decamethylcyclopentasiloxane).

The liquid fatty phase may additionally comprise additional oils and/or solvents which may be chosen from, alone or as a mixture:
fluorinated oils such as perfluoropolyethers, perfluoroalkanes, such as perfluorodecalin, perfluorodamantanes, monoesters, diesters and triesters of perfluoroalkyl phosphates and fluorinated ester oils;
oils of animal origin;
C6 to C40, in particular C10-C40, ethers; propylene glycol ethers that are liquid at room temperature, such as propylene glycol monomethyl ether, propylene glycol monomethyl ether acetate, dipropylene glycol mono-n-butyl ether;
C₈-C₃₂ fatty acids, such as oleic acid, linoleic acid, linolenic acid and mixtures thereof;
bifunctional oils, comprising two functional groups chosen from an ester and/or an amide and comprising from 6 to 30 carbon atoms, in particular 8 to 28 carbon atoms, even better from 10 to 24 carbons, and 4 heteroatoms chosen from O and N; preferably amide and ester functional groups being in the chain;
ketones that are liquid at room temperature (25° C.) such as methyl ethyl ketone, methyl isobutyl ketone, diisobutyl ketone, isophorone, cyclohexanone, acetone;
aldehydes that are liquid at room temperature, such as benzaldehyde, acetaldehyde.

The liquid fatty phase may represent 1 to 90% by weight of the composition, in particular 5 to 75% by weight, in particular 10 to 60% by weight, or even 25 to 55% by weight, of the total weight of the composition.

The composition according to the invention may advantageously comprise a thickening agent which may be chosen in particular from:
silicas, in particular hydrophobic silicas, such as those described in the document EP-A-898960, and for example marketed under the references “AEROSIL R812®” by the company Degussa, “CAB-O-SIL TS-530®”, “CAB-Q-SIL TS-610®”, “CAB-O-SIL TS-720®” by the company Cabot, “AEROSIL R972®”, “AEROSIL R974®” by the company Degussa;
clays such as montmorillonite, modified clays such as bentones for example, stearalkonium hectorite, stearalkonium bentonite,
alkyl ethers of polysaccharides (in particular whose alkyl group contains from 1 to 24 carbon atoms, preferably from 1 to 10, even better from 1 to 6, and more especially from 1 to 3) such as those described in the document EP-A-898958.

The quantity of thickening agent in the composition according to the invention may range from 0.05 to 40% by weight, relative to the total weight of the composition, preferably from 0.5 to 20% and even better from 1 to 15% by weight.

The composition according to the invention may also comprise at least one wax of plant, animal, mineral or synthetic origin, or even silicone wax.

There may be mentioned, in particular, alone or as a mixture, hydrocarbon waxes such as beeswax, Carnauba wax, Candelilla wax, ouricoury wax, Japan wax, cork fibre wax or sugarcane wax; paraffin wax, lignite wax, microcrystalline waxes; lanolin wax; Montan wax; ozokerites; polyethylene waxes; waxes obtained by Fischer-Tropsch synthesis; hydrogenated oils, fatty esters and glycerides that are concrete at 25° C. It is also possible to use silicone waxes, among which there may be mentioned alkyl polymethylsiloxane, alkoxy polymethylsiloxane and/or polymethylsiloxane esters.

The quantity of wax in the composition according to the invention may range from 0.1 to 70% by weight, relative to the total weight of the composition, preferably from 1 to 40% by weight, and even better from 5 to 30% by weight.

The composition according to the invention may also comprise one or more colouring matters chosen from pulverulent compounds such as pigments, fillers, pearlescent agents and glitter, and/or fat-soluble or water-soluble colorants.

The colouring matter, in particular the pulverulent colouring matter, may be present in the composition in an amount of 0.01 to 50% by weight, relative to the weight of the composition, preferably 0.1 to 40% by weight, or even 1 to 30% by weight.

The expression pigments should be understood to mean particles of any shape, that are white or coloured, inorganic or organic, insoluble in physiological medium, intended to colour the compositions.

The expression pearlescent agents should be understood to mean iridescent particles of any shape, in particular produced by certain molluscs in their shell or else synthesized.

The pigments may be white or coloured, inorganic and/or organic, interferential or otherwise. There may be mentioned, among the inorganic pigments, titanium dioxide, optionally surface-treated, zirconium or cerium oxides, and iron or chromium oxides, manganese violet, ultramarine blue, chromium hydrate and ferric blue. Among the organic pigments, there may be mentioned carbon black, D&C type pigments, and carmine-, barium-, strontium-, calcium and aluminium-based lacquers.

The pearlescent pigments may be chosen from white pearlescent pigments such as mica coated with titanium or with bismuth oxychloride, coloured pearlescent pigments such as mica-titanium with iron oxides, mica-titanium with in particular ferric blue or chromium oxide, mica-titanium with an organic pigment of the abovementioned type and bismuth oxychloride-based pearlescent pigments.

The fillers may be inorganic or organic, lamellar or spherical. There may be mentioned talc, mica, silica, kaolin, nylon and polyethylene powders, poly-β-alanine and polyethylene powders, Teflon, lauroyllysine, starch, boron nitride, tetrafluoroethylene polymer powders, hollow microspheres such as Expancel (Nobel Industrie), polytrap (Dow Corning) and microbeads of silicone resin (Tospearls from Toshiba, for example), precipitated calcium carbonate, magnesium carbonate and hydrocarbonate, hydroxyapatite, hollow silica microspheres (SILICA BEADS from MAPRECOS), glass or ceramic microcapsules, metal soaps derived from organic carboxylic acids having from 8 to 22 carbon atoms, preferably from 12 to 18 carbon atoms, for example zinc stearate, magnesium stearate or lithium stearate, zinc laurate, magnesium myristate.

The fat-soluble colorants are for example Sudan red, DC Red 17, DC Green 6, β-carotene, soybean oil, Sudan brown, DC Yellow 11, DC Violet 2, DC Orange 5, quinoline yellow. They may represent 0.01 to 20% of the weight of the composition, and even better from 0.1 to 6%.

The water-soluble colorants are, for example, beet juice, methylene blue, and may represent 0.01 to 6% of the total weight of the composition.

The composition may additionally comprise other ingredients commonly used in cosmetic compositions. Such ingredients may be chosen from antioxidants, perfumes, essential oils, preservatives, cosmetic active agents, moisturizers, vitamins, ceramides, sunscreens, surfactants, spreading agents, wetting agents, dispersing agents, antifoams, neutralizers, stabilizers, polymers and in particular fat-soluble film-forming polymers, and mixtures thereof.

Of course, persons skilled in the art will be careful to choose this or these optional additional compounds, and/or their quantity, such that the advantageous properties of the composition for use according to the invention are not, or not substantially, impaired by the addition envisaged.

The compositions according to the invention may be provided in any acceptable and customary form for a cosmetic or pharmaceutical composition.

They may therefore be provided in the form of a suspension, a dispersion, in particular an oil-in-water dispersion by means of vesicles; an organic or oily solution that is optionally thickened or even gelled; an oil-in-water, water-in-oil or multiple emulsion; a gel or a foam; an oily or emulsified gel; a dispersion of vesicles, in particular lipid vesicles; a two-phase or multiphase lotion; a spray; a lotion, a cream, an ointment, a soft paste, an unguent, a cast or moulded solid, and in particular as a stick or in a dish, or as a compact solid.

Persons skilled in the art will be able to choose the appropriate galenic form, and its method of preparation, on the basis of their general knowledge, taking into account, on the one hand, the nature of the constituents used, in particular their solubility in the carrier, and, on the other hand, the application envisaged for the composition.

The compositions in accordance with the invention have improved gloss and staying power of the gloss compared with the state of the art, they can be used for caring for or making up keratin materials such as the hair, the skin, the eyelashes, the eyebrows, the nails, the lips, the scalp and more particularly for making up the lips, the eyelashes and/or the face.

They may therefore be provided in the form of a care and/or make-up product for the skin of the body or of the face, for the lips, the eyelashes, the eyebrows, the hair, the scalp or the nails; an anti-sun or a self-tanning product; a hair product, in particular for dyeing, conditioning and/or caring for the hair; they are advantageously provided in the form of a mascara, a lipstick, a lip gloss, a blusher or an eyeshadow, or a foundation.

The subject of the invention is also a method for the cosmetic treatment of keratin materials, in particular the skin of the body or of the face, the lips, the nails, the hair and/or the eyelashes, comprising the application to the materials of a cosmetic composition as defined above.

This method according to the invention makes it possible in particular to care for or make up the lips by applying a lipstick or lip gloss composition according to the invention.

The invention is illustrated in greater detail in the following examples.

### Method of Measuring the Viscosity

The viscosity at 80° C. or 110° C. of the polymer is measured with the aid of a cone plate viscometer of the BROOKFIELD CAP 1000+ type.

The appropriate cone plate is determined by persons skilled in the art on the basis of their general knowledge, in particular:
between 50 and 500 mPa·s, it is possible to use a cone 02
between 500 and 1000 mPa·s: cone 03
between 1000 and 4000 mPa·s: cone 05
between 4000 and 10 000 mPa·s: cone 06

### Example 1

### Synthesis of Pentaerythrityl Benzoate/Isophthalate/Isostearate/Stearate

20 g of benzoic acid, 210 g of stearic acid, 70 g of isostearic acid and 100 g of pentaerythritol are loaded into a reactor equipped with mechanical stirring, an argon inlet and a distillation system, and then the mixture is gradually heated, under a gentle argon stream, to 110-130° C. in order to obtain a homogeneous solution. The temperature is then gradually increased to 180° C. and it is maintained for about 2 hours. The temperature is again increased to 220° C. and it is maintained until an acid value of less than or equal to 1 is obtained, which takes about 11 hours. The mixture is cooled to a temperature of between 100 and 130° C. and then 100 g of isophthalic acid are introduced and the mixture is again gradually heated to 220° C. for about 11 hours.

450 g of pentaerythrityl benzoate/isophthalate/isostearate/stearate polycondensate are thus obtained in the form of a very thick oil.

The polycondensate has the following characteristics:
soluble at 50% by weight, at 70° C., in Parleam
acid value=7.1
η_{110° C.}=850 mPa·s
Mw=28 500
ratio between the number of moles of aromatic monocarboxylic acid and the number of moles of non-aromatic monocarboxylic acids: 0.166.

### Example 2

### Synthesis of Pentaerythrityl Behenate/Benzoate/Isophthalate/Isostearate

20 g of benzoic acid, 140 g of behenic acid, 140 g of isostearic acid and 100 g of pentaerythritol are loaded into a reactor equipped with mechanical stirring, an argon inlet and a distillation system, and then the mixture is gradually heated, under a gentle argon stream, to 110-130° C. in order to obtain a homogeneous solution. The temperature is then gradually increased to 180° C. and it is maintained for about 2 hours. The temperature is again increased to 220° C. and it is maintained until an acid value of less than or equal to 1 is obtained, which takes about 11 hours. The mixture is cooled to a temperature of between 100 and 130° C. and then 100 g of isophthalic acid are introduced and the mixture is again gradually heated to 220° C. for about 11 hours.

440 g of pentaerythrityl behenate/benzoate/isophthalate/isostearate polycondensate are thus obtained in the form of a very thick oil.

The polycondensate has the following characteristics:
soluble at 50% by weight, at 70° C., in Parleam
acid value=4.2
η_{110° C.}=2050 mPa·s
ratio between the number of moles of aromatic monocarboxylic acid and the number of moles of non-aromatic monocarboxylic acids: 0.181.

### Example 3

In a manner similar to the preceding examples, the following polycondensates are prepared (the % values are by weight):

| | | | Polycarboxylic | | |
|---|---|---|---|---|---|
| | Polyol | Aromatic acid | acid or anhydride | Non-aromatic acids | |
| | (% and nature) | (% and nature) | (% and nature) | (% and nature) | Solubility * |
| Example A | 20.4% | 4.1% | 18.3% | 28.6% | at 25° C. |
| | penta- | benzoic | isophthalic | isostearic + | |
| | erythritol | | acid | 14.3% | |
| | | | | isononanoic + | |
| | | | | 14.3% stearic | |
| Example B | 20% | 4% | 20% | 18% | at 25° C. |
| | penta- | benzoic | isophthalic | isostearic + | |
| | erythritol | | acid | 38% stearic | |
| Example C | 20% | 4% | 20% | 28% | at 25° C. |
| | penta- | benzoic | isophthalic | isostearic + | |
| | erythritol | | acid | 28% stearic | |
| Example D | 19.8% | 4% | 19.8% | 40.6% | at 25° C. |
| | penta- | benzoic | isophthalic | isostearic + | |
| | erythritol | | acid | 15.8% stearic | |
| Example E | 19.8% | 4% | 19.8% | 48.5% | at 25° C. |
| | penta- | benzoic | isophthalic | isostearic + | |
| | erythritol | | acid | 7.9% stearic | |
| Example F | 19.8% | 4% | 19.8% | 52.4% | at 25° C. |
| | penta- | benzoic | isophthalic | isostearic + | |
| | erythritol | | acid | 4% stearic | |
| Example G | 25.5% | 3.9% | 15.7% | 34.9% | at 25° C. |
| | diglycerol | benzoic | sebacic acid | isostearic + | |
| | | | | 20% lauric | |
| Example H | 25% | 2.1% | 14.6% | 18.3% | at 70° C. |
| | trimethylol | m-toluic | phthalic | isostearic + | |
| | propane | | anhydride | 40% behenic | |
| Example I | 21.9% | 6.3% | 13.5% | 8.3% | at 70° C. |
| | erythritol | tert- | sebacic acid | isooctanoic + | |
| | | butyl | | 50% stearic | |
| | | benzoic | | | |
| Example J | 20.7% | 8.5% | 15.9% | 45.9% | at 25° C. |
| | glycerol | tert- | adipic acid | isononanoic + | |
| | | butyl | | 9% behenic | |
| | | benzoic | | | |
| Example K | 20% | 4% | 20% | 28% | at 70° C. |
| | penta- | benzoic | isophthalic | isostearic + | |
| | erythritol | | acid | 28% behenic | |

* “at 25° C.” indicates that the polymer is soluble at 50% by weight, at 25° C., in Parleam; “at 70° C.” indicates that the polymer is soluble at 50% by weight, at 70° C., in Parleam.

### Example 4

A lipstick having the following composition was prepared:

| | | |
|---|---|---|
| polycondensate of Example C | 30 | g |
| polyethylene wax | 11 | g |
| pigments and fillers | 7 | g |
| Parleam (hydrogenated isoparaffin) | qs 100 | g |

### Example 5

A lipstick having the following composition was prepared:

| | | |
|---|---|---|
| polycondensate of Example D | 30 | g |
| polyethylene wax | 11 | g |
| pigments and fillers | 7 | g |
| Parleam (hydrogenated isoparaffin) | qs 100 | g |

### Example 6

A lipstick having the following composition is prepared:

| | | |
|---|---|---|
| polycondensate of Example 1 | 10 | g |
| polyethylene wax | 11 | g |
| pigments and fillers | 7 | g |
| Parleam (hydrogenated isoparaffin) | qs 100 | g |

The above written description of the invention provides a manner and process of making and using it such that any person skilled in this art is enabled to make and use the same, this enablement being provided in particular for the subject matter of the appended claims, which make up a part of the original description and including compositions, optionally cosmetic and pharmaceutical compositions, comprising, optionally but preferably in a cosmetically or pharmaceutically acceptable medium, at least one polycondensate capable of being obtained, or obtained, by the reaction:
of 10 to 30% by weight, relative to the total weight of the polycondensate, of at least one polyol comprising 3 to 6 hydroxyl groups;
of 22 to 80% by weight, relative to the total weight of the polycondensate, of at least one linear, branched and/or cyclic, saturated or unsaturated, non-aromatic monocarboxylic acid comprising 10 to 32 carbon atoms, and having a melting point greater than or equal to 25° C.;
of 0.1 to 35% by weight, relative to the total weight of the polycondensate, of at least one linear, branched and/or cyclic, saturated or unsaturated, non-aromatic monocarboxylic acid comprising 6 to 32 carbon atoms and having a melting point strictly less than 25° C.;
of 0.1 to 10% by weight, relative to the total weight of the polycondensate, of at least one aromatic monocarboxylic acid comprising 7 to 11 carbon atoms, optionally additionally substituted with 1 to 3 linear, branched and/or cyclic, saturated or unsaturated alkyl radicals which comprise 1 to 32 carbon atoms;
of 1 to 40% by weight, relative to the total weight of the polycondensate, of at least one linear, branched and/or cyclic, saturated or unsaturated, or even aromatic, polycarboxylic acid comprising at least 2 carboxyl groups COOH, which may comprise heteroatoms; and/or a cyclic anhydride of such a polycarboxylic acid; and/or a lactone comprising at least one COOH group.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising at least one polycondensate obtained by the reaction:
of 10 to 30% by weight, relative to the total weight of the polycondensate, of at least one polyol comprising 3 to 6 hydroxyl groups;
of 22 to 80% by weight, relative to the total weight of the polycondensate, of at least one linear, branched and/or cyclic, saturated or unsaturated, non-aromatic monocarboxylic acid comprising 10 to 32 carbon atoms, and having a melting point greater than or equal to 25° C.;
of 0.1 to 35% by weight, relative to the total weight of the polycondensate, of at least one linear, branched and/or cyclic, saturated or unsaturated, non-aromatic monocarboxylic acid comprising 6 to 32 carbon atoms and having a melting point less than 25° C.;
of 0.1 to 10% by weight, relative to the total weight of the polycondensate, of at least one aromatic monocarboxylic acid comprising 7 to 11 carbon atoms, optionally additionally substituted with 1 to 3 linear, branched and/or cyclic, saturated or unsaturated alkyl radicals which comprise 1 to 32 carbon atoms;
of 1 to 40% by weight, relative to the total weight of the polycondensate, of at least one linear, branched and/or cyclic, saturated or unsaturated, or aromatic, polycarboxylic acid comprising at least 2 carboxyl groups COOH, which may comprise heteroatoms; and/or a cyclic anhydride of such a polycarboxylic acid; and/or a lactone comprising at least one COOH group.

2. The composition according to claim 1, wherein the polyol is chosen from, alone or as a mixture, 1,2,4-butanetriol, 1,2,6-hexanetriol, trimethylolethane, trimethylolpropane, glycerol pentaerythritol, erythritol, diglycerol, ditrimethylolpropane, xylitol, sorbitol, mannitol dipentaerythritol, and triglycerol.

3. The composition according to claim 1, wherein the polyol, or the mixture of polyols, represents 12 to 25% by weight of the total weight of the polycondensate.

4. The composition according to claim 1, wherein the non-aromatic monocarboxylic acid having a melting point greater than or equal to 25° C. is chosen from, alone or as a mixture, decanoic acid, lauric acid, tridecanoic acid, myristic acid, palmitic acid, stearic acid, arachidonic acid, behenic acid, and cerotic (hexacosanoic) acid, petroselinic acid, vaccenic acid, elaidic acid, gondoic acid, gadoleic acid, erucic acid, and nervonic acid.

5. The composition according to claim 1, wherein the non-aromatic monocarboxylic acid having a melting point greater than or equal to 25° C., or the mixture of the acids, represents 25 to 75% by weight of the total weight of the polycondensate.

6. The composition according to claim 1, wherein the non-aromatic monocarboxylic acid having a melting point of less than 25° C. is chosen from, alone or as a mixture, caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, isononanoic acid, isostearic acid, caproleic acid, obtusilic acid, undecylenic acid, dodecylenic acid, linderic acid, myristoleic acid, physeteric acid, tsuzuic acid, palmitoleic acid, oleic acid, linoleic acid, linolenic acid, and arachidonic acid.

7. The composition according to claim 1, wherein the non-aromatic monocarboxylic acid having a melting point of less than 25° C. represents 0.5 to 32% by weight of the total weight of the polycondensate.

8. The composition according to claim 1, wherein the aromatic monocarboxylic acid is chosen from benzoic acid, o-toluic acid, m-toluic acid, p-toluic acid, 1-naphthoic acid, 2-naphthoic acid, 4-tert-butylbenzoic acid, 1-methyl-2-naphthoic acid, 2-isopropyl-1-naphthoic acid, and mixtures thereof.

9. The composition according to claim 1, wherein the aromatic monocarboxylic acid represents 0.5 to 9.95% by weight of the total weight of the polycondensate.

10. The composition according to claim 1, wherein the polycarboxylic acid, its anhydride or the lactone, is chosen from, alone or as a mixture:
saturated or unsaturated, or even aromatic, linear, branched and/or cyclic polycarboxylic acids comprising 2 to 50 carbon atoms; the acid comprising at least two carboxyl groups COOH; and optionally capable of comprising 1 to 10 heteroatoms, which are identical or different, chosen from O, N and S; and/or optionally capable of comprising at least one perfluorinated radical chosen from —CF₂— (divalent) or —CF₃; and their anhydrides;
polycarboxylic acids having a linear or branched, saturated or unsaturated chain comprising at least one heteroatom chosen from O, N and/or S, and/or comprising at least one perfluorinated radical —CF₂— or —CF₃ and having moreover at least 2 carboxyl groups COOH; and their anhydrides;
saturated, unsaturated, or aromatic heterocyclic polycarboxylic acids comprising at least one heteroatom chosen from O, N and/or S and at least 2 carboxyl groups COOH; and their anhydrides;
polycarboxylic acids derived from a sugar and comprising at least 2 carboxyl groups COOH; and their anhydrides;
itaconic anhydride and the 1,4-monoanhydride of 1,4,5,8-naphthalenetetracarboxylic acid;
polycarboxylic acids having a linear, branched and/or cyclic, saturated or unsaturated chain optionally comprising at least one heteroatom chosen from O, N and/or S and/or optionally comprising at least one perfluorinated radical —CF₂— or —CF₃; and additionally comprising at least one primary, secondary or tertiary amine functional group functional group and having moreover at least 2 carboxyl groups COOH; and their anhydrides;
lactones comprising 1, 2 or 3 COOH groups, and 5 to 14 carbon atoms.

11. The composition according to claim 1, wherein the polycarboxylic acid and/or its cyclic anhydride and/or the lactone represents 5 to 35% by weight of the total weight of the final polycondensate.

12. The composition according to claim 1, wherein the composition further comprises a cosmetically or pharmaceutically acceptable medium, and wherein the polycondensate is present in a quantity of between 0.1 and 70% by weight relative to the weight of the final composition.

13. The composition according to claim 1, wherein said composition is in the form of a care and/or make-up product for the skin of the body or of the face, the lips, the eyelashes, the eyebrows, the hair, the scalp or the nails; an anti-sun or a self-tanning product; or a hair product for dyeing, conditioning and/or caring for the hair.

14. Method for the treatment of a keratin material, comprising the application to the material of the composition defined in claim 1.

15. A polycondensate obtained by the reaction:
of 10 to 30% by weight, relative to the total weight of the polycondensate, of at least one polyol comprising 3 to 6 hydroxyl groups;
of 22 to 80% by weight, relative to the total weight of the polycondensate, of at least one linear, branched and/or cyclic, saturated or unsaturated, non-aromatic monocarboxylic acid comprising 10 to 32 carbon atoms, and having a melting point greater than or equal to 25° C.;
of 0.1 to 35% by weight, relative to the total weight of the polycondensate, of at least one linear, branched and/or cyclic, saturated or unsaturated, non-aromatic monocarboxylic acid comprising 6 to 32 carbon atoms and having a melting point less than 25° C.;
of 0.1 to 10% by weight, relative to the total weight of the polycondensate, of at least one aromatic monocarboxylic acid comprising 7 to 11 carbon atoms, optionally additionally substituted with 1 to 3 linear, branched and/or cyclic, saturated or unsaturated alkyl radicals which comprise 1 to 32 carbon atoms;
of 1 to 40% by weight, relative to the total weight of the polycondensate, of at least one linear, branched and/or cyclic, saturated or unsaturated, or even aromatic, polycarboxylic acid comprising at least 2 carboxyl groups COOH, which may comprise heteroatoms; and/or a cyclic anhydride of such a polycarboxylic acid; and/or a lactone comprising at least one COOH group.

16. A method of preparing a polycondensate according to claim 15, comprising:
mixing the polyol and the aromatic and non-aromatic monocarboxylic acids to form a mixture,
heating the mixture under an inert atmosphere, first up to the melting point, and then to a temperature of between 150 and 220° C. until there is complete consumption of the monocarboxylic acids, to form a heated mixture and then
optionally cooling the heated mixture to a temperature of between 90 and 150° C.,
adding the polycarboxylic acid and/or the cyclic anhydride and/or the lactone to the heated mixture, optionally after cooling, to produce a third mixture and then
heating the third mixture to a temperature of less than or equal to 220° C.
